(19) United States
(12) Patent Application Publication
(10) Pub. No.: US 2026/0162773 A1
(43) Pub. Date: Jun. 11, 2026

(54) Synthetic Genetic Code Management System Utilizing Blockchain

(71) Applicants: Tamara Pack, Wrightwood, CA (US); Maria M Harney, Wrightwood, CA (US)

(72) Inventors: Tamara Pack, Wrightwood, CA (US); Maria M Harney, Wrightwood, CA (US)

(21) Appl. No.: 18/976,169

(22) Filed: Dec. 10, 2024

(51) Int. Cl. G16B 40/30; G06F 21/10; G16B 50/30

(52) U.S. Cl. CPC G16B 40/30; G06F 21/101; G16B 50/30

(57) ABSTRACT

A system for managing synthetic genetic codes comprising an artificial intelligence (AI) component, a blockchain component, and a data storage component is provided. The AI component analyzes genetic data using deep learning algorithms. The blockchain component records ownership and transactions related to synthetic genetic constructs. The data storage component stores digital models of DNA sequences. The system utilizes Graph Convolutional Networks, Convolutional Neural Networks, or Recurrent Neural Networks. The system secures DNA models using blockchain technology. The system provides a method for managing synthetic genetic codes. The method comprising analyzing genetic data using an AI component, recording transactions using a blockchain component, and storing DNA models using a data storage component, predicting potential applications of synthesized genetic codes, validating synthetic genetic constructs, transforming genetic sequences into digital assets, generating utility tokens on a blockchain network, and eliminating biases in AI algorithms.

### FIELD OF THE INVENTION

The present disclosure relates to systems and methods for managing synthetic genetic codes and biological systems. More specifically, embodiments of this present disclosure relate to one or more of the following CPC classifications: G16H50/20 ICT specially adapted for medical diagnosis, medical simulation or medical data mining; ICT specially adapted for detecting, monitoring or modelling epidemics or pandemics for computer-aided diagnosis, e.g. based on medical expert systems; G06F16/2465 Query processing support for facilitating data mining operations in structured databases; G06F16/9024 Graphs; Linked lists; G06N20/00 Machine learning; G06N20/10 Machine learning using kernel methods, e.g. support vector machines [SVM]; G06N20/20 Ensemble learning; G06N3/084 Backpropagation, e.g. using gradient descent; G06N5/02 Knowledge representation; Symbolic representation; G06Q20/065 Private payment circuits, e.g. involving electronic currency used among participants of a common payment scheme using e-cash; G06Q20/308 Payment architectures, schemes or protocols characterised by the use of specific devices or networks using the Internet of Things; G06Q30/0206 Price or cost determination based on market factors; G16H10/40 ICT specially adapted for the handling or processing of patient-related medical or healthcare data for data related to laboratory analysis, e.g. patient specimen analysis; G16H20/10 ICT specially adapted for therapies or health-improving plans, e.g. for handling prescriptions, for steering therapy or for monitoring patient compliance relating to drugs or medications, e.g. for ensuring correct administration to patients; G16H20/30 ICT specially adapted for therapies or health-improving plans, e.g. for handling prescriptions, for steering therapy or for monitoring patient compliance relating to physical therapies or activities, e.g. physiotherapy, acupressure or exercising; G16H20/40 ICT specially adapted for therapies or health-improving plans, e.g. for handling prescriptions, for steering therapy or for monitoring patient compliance relating to mechanical, radiation or invasive therapies, e.g. surgery, laser therapy, dialysis or acupuncture; G16H20/60 ICT specially adapted for therapies or health-improving plans, e.g. for handling prescriptions, for steering therapy or for monitoring patient compliance relating to nutrition control, e.g. diets; G16H40/67 ICT specially adapted for the management or administration of healthcare resources or facilities; ICT specially adapted for the management or operation of medical equipment or devices for the operation of medical equipment or devices for remote operation; G16H50/30 ICT specially adapted for medical diagnosis, medical simulation or medical data mining; ICT specially adapted for detecting, monitoring or modelling epidemics or pandemics for calculating health indices; for individual health risk assessment; G16H50/70 ICT specially adapted for medical diagnosis, medical simulation or medical data mining; ICT specially adapted for detecting, monitoring or modelling epidemics or pandemics for mining of medical data, e.g. analysing previous cases of other patients; G06F2216/03 Data mining; G06N3/045 Combinations of networks; G06N5/01 Dynamic search techniques; Heuristics; Dynamic trees; Branch-and-bound; G06N5/04 Inference or reasoning models; G06N7/01 Probabilistic graphical models, e.g. probabilistic networks; G06N7/023 Learning or tuning the parameters of a fuzzy system.

### BACKGROUND OF THE INVENTION

The present disclosure relates to systems and methods for managing synthetic genetic codes and biological systems. Synthetic biology and genetic engineering may represent rapidly advancing fields with potential applications in medicine, agriculture, and industrial biotechnology. The manipulation and creation of genetic codes may involve complex processes that may require sophisticated analysis and management systems. Conventional approaches to managing synthetic genetic codes may face challenges related to data security, intellectual property protection, and efficient analysis of genetic information.

Traditional methods for handling genetic data may rely on separate systems for sequencing, analysis, and data storage. This fragmented approach may lead to inefficiencies and potential security vulnerabilities. For example, genetic sequence data may be stored in one database, while analysis results may be kept in another, potentially increasing the risk of data breaches or unauthorized access.

Biosafety concerns may arise from the potential misuse or accidental release of synthetic genetic constructs. Conventional safety protocols may rely heavily on physical containment and access restrictions. However, these measures may not always account for the digital nature of genetic information and the potential for unauthorized replication or modification. Furthermore, the storage and management of synthetic genetic codes may pose unique challenges. Traditional data storage solutions may not be optimized for the specific requirements of genetic data, which may include long-term stability, quick retrieval, and secure sharing among authorized parties. This may lead to inefficiencies in data management and potential risks to data integrity. There is a need for improved systems of safeguarding and managing genetic information.

### SUMMARY

A system for managing synthetic genetic codes is provided. The system for managing synthetic genetic codes may include an artificial intelligence (AI) component, a blockchain component, and a data storage component. system for managing synthetic genetic codes may be configured to provide intellectual property management in synthetic biology may present unique challenges.

In one or more embodiments, the system for managing synthetic genetic codes may provide for more efficient means of maintaining confidentiality while providing real-time protection or transparency in rapidly evolving research environments. In one or more embodiments, the system for managing synthetic genetic codes may provide researchers with clear ownership and track the use of synthetic genetic constructs.

The system for managing genetic codes may comprise an AI component. The AI component may analyze genetic data using deep learning algorithms. The AI component may be configured to provide analysis of genetic data involving complex computational methods. The system for managing genetic codes may provide advanced bioinformatics tools with robust processing allowing for interpreting large volumes of genetic information efficiently. In one or more embodiments, the system for managing genetic codes may comprise a blockchain component may record ownership and transactions related to synthetic genetic constructs. The system for managing genetic codes may further comprise a data storage component may store digital models of DNA sequences.

These together with additional objects, features and advantages of the system for managing synthetic genetic codes will be readily apparent to those of ordinary skill in the art upon reading the following detailed description of the presently preferred, but nonetheless illustrative, embodiments when taken in conjunction with the accompanying drawings. Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are and explanatory only and are not restrictive of the disclosure, as claimed.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

The accompanying drawings, which are incorporated in and constitute a part of this specification, illustrate embodiments of the disclosure and, together with a general description of the disclosure given above, and the detailed description given below, serve to explain the principles of the disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a high-level block diagram illustrating machine learning model inference in accordance with some embodiments.

FIGS. 2A AND 2B illustrates a synthetic genetic code management system utilizing blockchain in accordance with some embodiments.

FIG. 3 illustrates a synthetic genetic code management system utilizing blockchain in accordance with some embodiments.

FIGS. 4A AND 4B illustrates a synthetic genetic code management system utilizing blockchain in accordance with some embodiments.

FIGS. 5A AND 5B is a high-level block diagram illustrating a system diagram in accordance with some embodiments.

FIG. 6 is an illustration of a high-level block diagram of the computing device of the system in accordance with some embodiments.

FIG. 7A may be a flowchart illustrating a process for genetic data analysis and modification.

FIG. 7B may be a flowchart depicting three main processes: blockchain integration, verification and validation, and access and distribution.

FIG. 8A may illustrate a simple example of a Synthetico genetic patch.

FIG. 8B may illustrate an example of using Synthetico's proprietary AI to address genetic disorders, specifically Trisomy 18 (Edwards syndrome).

### DETAILED DESCRIPTION

The present disclosure includes many aspects and features. Moreover, while many aspects and features relate to, and are described in, the context of systems and methods of fraud identification, embodiments of the present disclosure are not limited to use only in this context. The present disclosure can be understood more readily by reference to the following detailed description of the disclosure and the examples included therein.

Before the present articles, systems, apparatuses, and/or methods are disclosed and described, it is to be understood that they are not limited to specific methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise. As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

It is understood that the apparatuses and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

The following description of various embodiments is merely exemplary in nature and is in no way intended to limit the disclosure, its application, or uses.

A system for managing synthetic genetic codes and biological systems may be referred to as Synthetico. In one or more embodiments, the system may include three main components: an AI software component, a blockchain component, and a data storage component. The AI software component may employ deep learning techniques. These techniques may include Graph Convolutional Networks, Convolutional Neural Networks (2D and 3D), Recurrent Neural Networks, and 1D Convolutional Networks.

In one or more embodiments, the AI component may process genetic data generated by sequencers in laboratories. It may interact with the blockchain system to record and manage genetic information. The blockchain component may serve as a framework for transaction and record-keeping. It may include a blockchain plugin for laboratories and sequencers. The blockchain component may enable real-time recording of intellectual property rights associated with new genetic creations. The data storage component may include dedicated servers for storing digital models of DNA sequences. These models may be secured by blockchain technology.

In one or more embodiments, the system may operate within a technical environment encompassing deep learning AI, genetic sequencing, and blockchain technology. It may be designed for use in laboratory settings where genetic codes and sequences are synthesized and analyzed. The AI software may be implemented on high-performance computing systems capable of handling complex deep learning tasks. The blockchain infrastructure may comprise nodes, network infrastructure, and plugins specifically designed for integration with laboratory equipment including AI plugins. In one or more instances, the plugins include but are not limited to one or more of the following: development and productivity plugins, web and UI design plugins, AI-powered plugins, blockchain plugins, data analysis and visualization plugins, DevOps and automation plugins, content and media plugins, e-commerce and marketing plugins, and security and monitoring plugins.

The data storage systems may be high-capacity, secure servers designed to store and manage large volumes of genetic data and associated intellectual property information. From the perspective of a computing device handling the AI software, the system may analyze genetic information using deep learning algorithms. It may predict potential applications of synthesized genetic codes and interface with the blockchain to record transactions. From the perspective of the blockchain network, the system may process transactions, record ownership and licenses of genetic creations, and ensure data integrity through cryptographic techniques.

From the perspective of a laboratory technician, the system may be used to input genetic data and monitor the synthesis process. The technician may interact with the AI for predictive modeling and validation of synthetic genetic constructs. From the perspective of a regulatory body, the system may allow monitoring and auditing of the blockchain ledger to ensure compliance with biosafety and intellectual property regulations.

In one or more embodiments, the system may transform genetic sequences into intellectual property. Genetic data, once synthesized and validated by AI algorithms, may be transformed into a secure digital asset represented by a blockchain token. This token may signify ownership and rights to the synthesized genetic construct, enabling management of intellectual property. The system may use a novel predictive modeling platform. This platform may utilize proprietarily trained AI combined with deep learning structures. The deep learning structures may include a Graph convolutional network, a convolutional neural network 2D and 3D, a Recurrent neural network, and a 1D convolutional network. These networks may utilize sequential, spatial, and image data synergistically.

In one or more embodiments, the system may employ neural networks, a biologically-inspired programming paradigm which may enable a computer to learn from observational data. In one or more embodiments, the system may use deep learning, a set of techniques for learning in neural networks involving perceptron. Perceptron may be an artificial neuron, that may become a method for weighing evidence to make decisions. In one or more embodiments, the system may include a proprietary algorithm for how blockchain works and stores information.

In one or more embodiments, the system may have several controls and layers, with the innermost layer of genetic codes being accessible through a Synthetico coin purchase. Synthetico may be a digital security token backed by a real-world asset, such as newly synthesized genetic codes, sequences and biological systems. These assets may become the users'proprietary intellectual property via blockchain technology. In one or more embodiments, the system may generate utility tokens at ICO on its native blockchain network. These utility coins may allow for services to the user. Each Synthetico unit generated and issued into circulation may be backed by one singular and definitive biological piece of synthesized genetic material which may have no prior existence in nature. In one or more embodiments, the system may store models of DNA, which may save storage space. The Synthetico AI software may act as a liaison between individuals/laboratories and the blockchain.

In one or more embodiments, the system may include built-in controls, including AI on AI and human controls, bias elimination systems, and quantum-proof algorithms. The system may be trained using various sources and robust predictive mechanisms to look for pre-selected patterns. The system may account for biases and their differentiation from pre-selected variables. AI algorithms may account for 3-4 times more neural layers and processes in comparison to current industry standards. In one or more embodiments, the system may transform DNA sequences into models for storage. It may create new DNA codes/products, proofread them, and assist in assessing their usefulness, viability, and non-harmfulness.

In one or more embodiments, the deep learning networks may be built using Python. In one or more embodiments, the system may employ proprietary AI algorithms. In one or more embodiments, for the Synthetico deep neural network topology/architecture, the system may stack and blend several ML algorithms/models. To classify various synthetic systems, the system may first employ a logistic regression algorithm to estimate binary values and solve classification problems of various synthesized categories. The system may then employ two models that may serve as a cross-checking control mechanism: linear regression and decision trees or random forests. Linear regression, a self-learning algorithm, may establish relationships between independent and dependent variables using a y=ax+b function. This model may establish the relationship between variables and biases in the hidden layers for continuous outcomes. Decision trees or random forests, non-parametric models, may separate information at different nodes based on different features/variables of the dataset. Outcomes from both models may be cross-checked and imputed into a final logistic regression control to decide on the outputs and initiate approval, disapproval, or send it to human control. In one or more embodiments, the system may produce three possible outcomes: yes, no, or maybe.

In one or more embodiments, the system may transform genetic sequences into intellectual property. In one or more embodiments, genetic data, once synthesized and validated by AI algorithms, may be transformed into a secure digital asset represented by a blockchain token. This token may signify ownership and rights to the synthesized genetic construct, enabling management of intellectual property. In one or more embodiments, the system may use a novel predictive modeling platform. This platform may utilize proprietarily trained AI combined with deep learning structures. In one or more embodiments, the deep learning structures may include a Graph convolutional network, a convolutional neural network 2D and 3D, a Recurrent neural network, and a 1D convolutional network. These networks may utilize sequential, spatial, and image data synergistically. In one or more embodiments, the system may employ neural networks, a biologically-inspired programming paradigm which may enable a computer to learn from observational data.

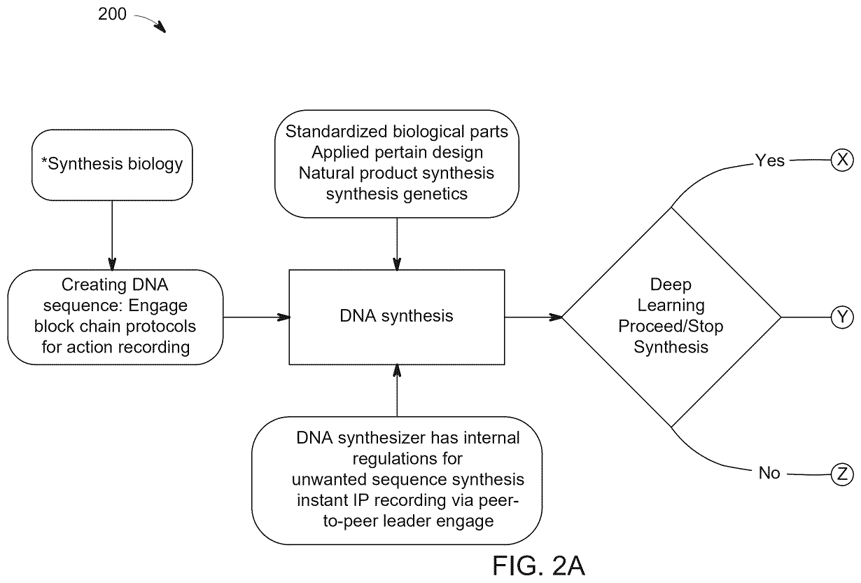

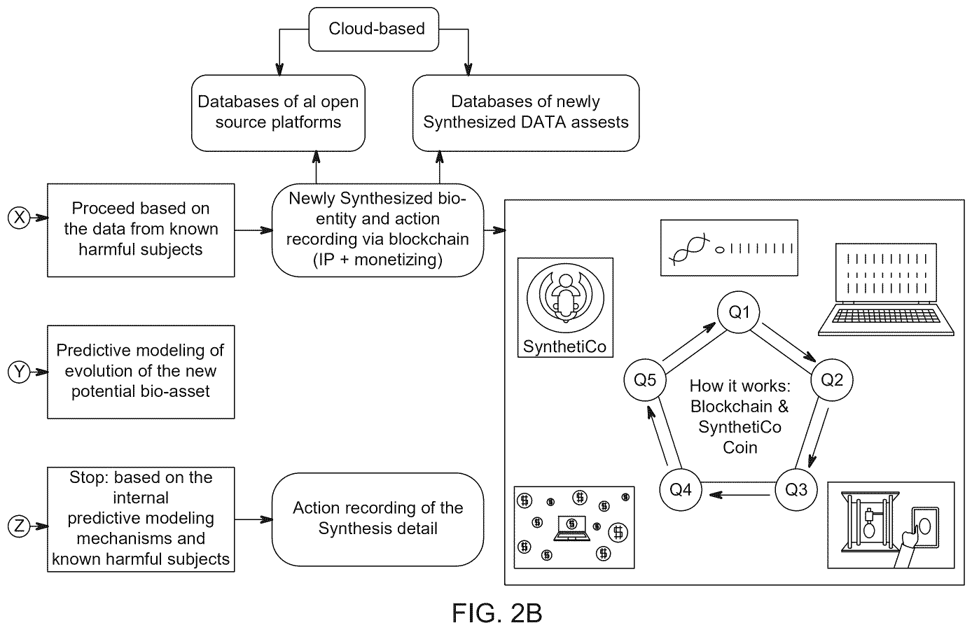

Regarding FIGS. 2A AND 2B, 260, the system may include a proprietary algorithm for how blockchain works and stores information. It may have several controls and layers, with the innermost layer of genetic codes being accessible through a Synthetico coin purchase. Synthetico may be a digital security token backed by a real-world asset, such as newly synthesized genetic codes, sequences and biological systems. These assets may become the users'proprietary intellectual property via blockchain technology. The system may generate utility tokens at ICO on its native blockchain network. These utility coins may allow for services to the user. In one or more embodiments, each Synthetico unit generated and issued into circulation may be backed by one singular and definitive biological piece of synthesized genetic material which may have no prior existence in nature. In one or more embodiments, the system may store models of DNA, which may save storage space. In one or more embodiments, the Synthetico AI software may act as a liaison between individuals/laboratories and the blockchain.

In one or more embodiments, the system may include built-in controls, including AI on AI and human controls, bias elimination systems, and quantum-proof algorithms. The system may be trained using various sources and robust predictive mechanisms to look for pre-selected patterns. In one or more embodiments, the system may account for biases and their differentiation from pre-selected variables. AI algorithms may account for 3-4 times more neural layers and processes in comparison to current industry standards.

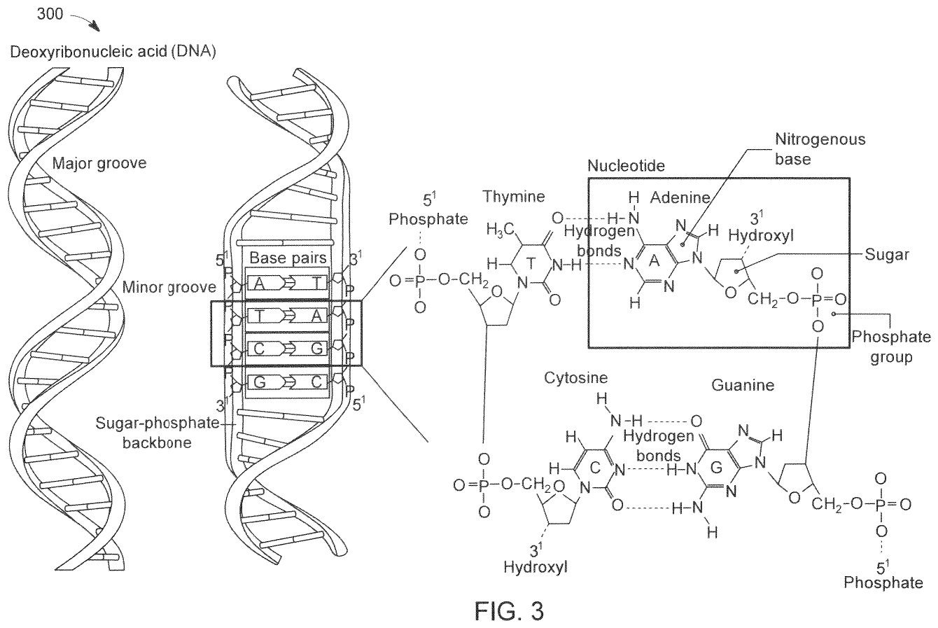

Regarding FIG. 3, 300, the system may transform DNA sequences into models for storage. It may create new DNA codes/products, proofread them, and assist in assessing their usefulness, viability, and non-harmfulness. In one or more embodiments, to classify various synthetic systems, the system may first employ a logistic regression algorithm to estimate binary values and solve classification problems of various synthesized categories. In one or more embodiments, the system may analyze genetic data using deep learning algorithms. It may predict potential applications of synthesized genetic codes and interface with the blockchain to record transactions. In one or more embodiments, the system may process transactions, record ownership and licenses of genetic creations, and ensure data integrity through cryptographic techniques. In one or more embodiments, the system may allow a laboratory technician to input genetic data and monitor the synthesis process. The technician may interact with the AI for predictive modeling and validation of synthetic genetic constructs. In one or more embodiments, the system may allow a regulatory body to monitor and audit the blockchain ledger to ensure compliance with biosafety and intellectual property regulations.

In one or more embodiments, the system may include a blockchain component that may serve as a framework for transaction and record-keeping. It may include a blockchain plugin for laboratories and sequencers. The blockchain component may enable real-time recording of intellectual property rights associated with new genetic creations. In one or more embodiments, the system may include a data storage component that may include dedicated servers for storing digital models of DNA sequences. These models may be secured by blockchain technology. In one or more embodiments, the system may operate within a technical environment encompassing deep learning AI, genetic sequencing, and blockchain technology. It may be designed for use in laboratory settings where genetic codes and sequences are synthesized and analyzed. In one or more embodiments, the AI software may be implemented on high-performance computing systems capable of handling complex deep learning tasks. The blockchain infrastructure may comprise nodes, network infrastructure, and plugins specifically designed for integration with laboratory equipment including AI plugins. In one or more instances, the plugins include but are not limited to one or more of the following: development and productivity plugins, web and UI design plugins, AI-powered plugins, blockchain plugins, data analysis and visualization plugins, DevOps and automation plugins, content and media plugins, e-commerce and marketing plugins, and security and monitoring plugins.

The data storage systems may be high-capacity, secure servers designed to store and manage large volumes of genetic data and associated intellectual property information. In one or more embodiments, the system may utilize blockchain technology, a distributed database that may act as a distributed trusted peer-to-peer ledger system for storing transactions. Each transaction may be encrypted and linked to the previous one through cryptographic signatures, ensuring immutability. In one or more embodiments, the system may include its native blockchain network, token, and coin. The Synthetico token may be created using ERC-20 protocol and Liquid Network for their efficiency, fast deployment, and scale capabilities. In one or more embodiments, the system may include proprietary code written on LAYER 0 PROTOCOL, which may allow integrated connectivity of various protocols. Quantum-resistant encryption algorithms may be integrated into blockchain technology ensuring secure transactions and data management. In one or more embodiments, the system may include a Synthetico/HERMES coin that may have its standalone independent native blockchain. This blockchain network may provide a peer-to-peer ledger and incentive for verification of the minted information and may contain encrypted genetic codes from new engineered biological sequences and circuits and more complex biological systems.

In one or more embodiments, the system may offer HERMES coins for initial pre-sales, where coins backed by the genetic codes created by the Synthetico AI platform may be offered to investors. In one or more embodiments, the system may include a Synthetico Utility token, a type of cryptocurrency created during Initial Coin Offerings (ICOs). Utility tokens may provide access to specific services or products within a HERMES network. The system may allow for trading Synthetico/HERMES coins on exchanges, which may generate additional revenues from transaction fees. The system may achieve advanced security, transparency and traceability, accessible global market, asset tokens, smart contract functionality, and scalability through its blockchain setup. The system may build on various protocols—ETH-20, Solana, Polygon or any other novel network [[ot]]or Blockchain.

In one or more embodiments, the system may have several layers of how it generates and stores information. The first one may be AI generated models and human DNA sequences that may be generated or recorded respectively and stored at the servers as models. DNA sequences may also be recorded on blockchain, and may be accessible by users once the author's permission is acquired. AI generated DNA sequences and newly synthesized DNA sequences from other sources may be transformed by AI software into models and stored on blockchain.

In one or more embodiments, the system may require robust storage solutions for the successful deployment of blockchain technology. The storage requirements for a blockchain network, both on-chain and off-chain, may be substantial and complex, especially considering regulations like GDPR. On-chain storage may involve storing ledger data, which may be immutable and may not be pruned. Off-chain storage may be necessary for supporting data like pictures, contracts, and personal information, which may not be stored directly on the blockchain ledger. For Hyperledger-based blockchains, the current performance may be approximately 3100 transactions per second (TPS), with a projected increase to as high as 30,000 TPS. A conservative estimate may suggest that at 100 TPS, storage requirements may be approximately 0.659 terabytes (TiB) per year, or at 205 TPS, it may reach 3.215 TiB per year. For each transaction, approximately 0.3 documents may be generated, resulting in significant off-chain storage requirements. The estimated storage required for off-chain documents may be approximately 271 gigabytes (GB) per TPS per year. Different blockchain implementations, transaction volume, data types, and retention policies may require varying amounts of storage, but proper data management, backup protocols, and replication may be critical for ensuring data integrity and compliance.

In one or more embodiments, the system may utilize blockchain technology, which may act as a distributed trusted peer-to-peer ledger system for storing transactions. Each transaction may be encrypted and linked to the previous one through cryptographic signatures, potentially ensuring immutability. The system may include its native blockchain network, token, and coin. The Synthetico token may be created using ERC-20 protocol and Liquid Network for their efficiency, fast deployment, and scale capabilities. The system may include proprietary code written on LAYER 0 PROTOCOL, which may allow integrated connectivity of various protocols. Quantum-resistant encryption algorithms may be integrated into blockchain technology potentially ensuring secure transactions and data management. The system may include a Synthetico/HERMES coin that may have its standalone independent native blockchain. This blockchain network may provide a peer-to-peer ledger and incentive for verification of the minted information and may contain encrypted genetic codes from new engineered biological sequences and circuits and more complex biological systems. In one or more embodiments, the system may offer HERMES coins for initial pre-sales, where coins backed by the genetic codes created by the Synthetico AI platform may be offered to investors. The system may include a Synthetico Utility token, a type of cryptocurrency created during Initial Coin Offerings (ICOs). Utility tokens may provide access to specific services or products within a HERMES network. The system may allow for trading Synthetico/HERMES coins on exchanges, which may generate additional revenues from transaction fees. The system may achieve advanced security, transparency and traceability, accessible global market, asset tokens, smart contract functionality, and scalability through its blockchain setup.

In one or more embodiments, different blockchain implementations, transaction volume, data types, and retention policies may require varying amounts of storage, but proper data management, backup protocols, and replication may be critical for ensuring data integrity and compliance. The system may include a data collection and storage component. This component may collect genetic data such as DNA sequences and mutations from various sources including organisms, samples, and databases. The collected genetic material may be stored securely in a centralized or distributed database. In one or more embodiments, the system may include a modeling and analysis component. This component may use machine learning and AI algorithms to model genetic behavior. It may analyze patterns, mutations, and interactions, and identify genes related to specific traits. The system may include a modification suggestion component. Based on user input, this component may suggest genetic modifications and optimize genes for desired traits. The output may be modified genetic sequences or recombinant DNA sequences. The system may include a blockchain integration component. This component may create a unique digital identity (hash) for each modified genetic sequence and record the hash on a blockchain. This may ensure immutability and transparency.

In one or more embodiments, the system may include a verification and validation component. This component may validate modifications against original genetic material, confirm accuracy and safety, and involve experts and peer review. The system may include an access and distribution component. This component may allow authorized users to access the modified genetic material and facilitate collaboration and research. The output may be shared and accessible genetic information that provides proof of creation and ownership, as well as the opportunity to monetize it. The system may include additional revenue streams. These may include an ATM machine that prints physical copies of Synthetico coins or exchanges crypto from a physical wallet to any currency. The system may also include merchandise such as crypto physical wallets with logos or unique designer pieces.

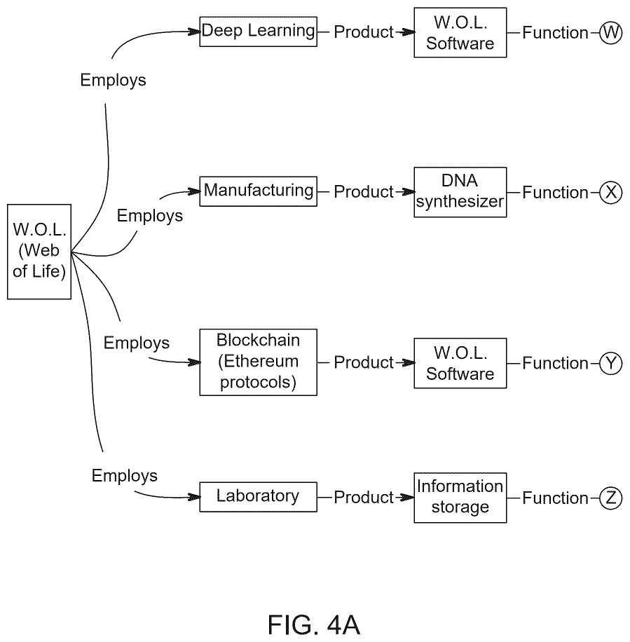

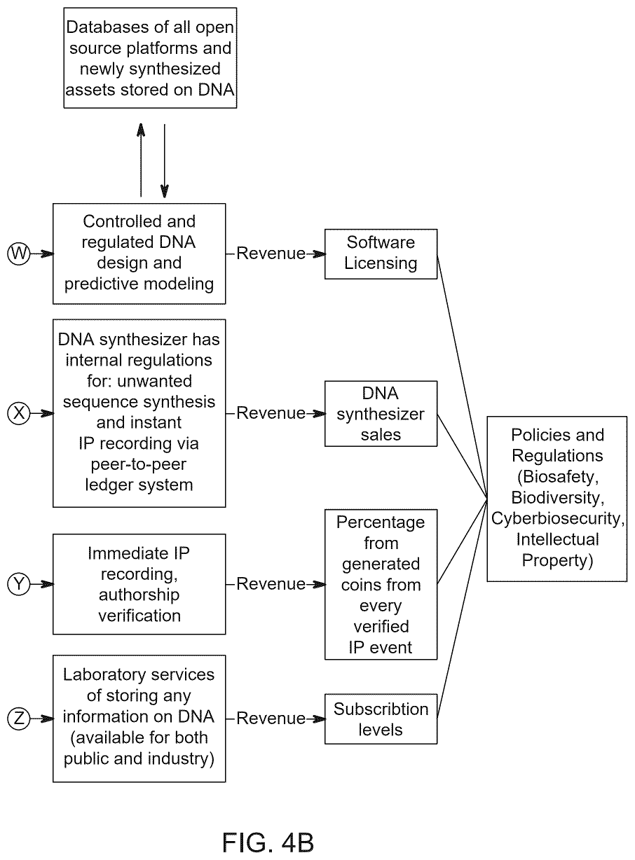

Regarding FIGS. 4A AND 4B, 400, the system may operate within a larger framework, potentially referred to as a “Web of Life” (W.O.L.). This framework may include deep learning and manufacturing components, leading to product creation. The products may interface with W.D.L. Software, which may connect to various elements such as DNA synthesizers and software licensing. The system may incorporate blockchain protection, potentially using Ethereum. It may interface with laboratories and information storage components, each potentially leading to product creation and interfacing with W.O.L. Software.

In one or more embodiments, the system may generate revenue streams through software licensing, percentages from revenues going into certified IP or events, and subscription levels. The system may operate within a framework of policies, preservation and regulations covering biosafety, biodiversity, cybersecurity, and intellectual property. In one or more embodiments, the system may include a genetic data collection and storage component. This component may collect genetic data such as DNA sequences and mutations from various sources including organisms, samples, and databases. The collected genetic material may be stored securely in a centralized or distributed database. In one or more embodiments, the system may include a modeling and analysis component. This component may use machine learning and AI algorithms to model genetic behavior. It may analyze patterns, mutations, and interactions, and identify genes related to specific traits. The system may include a modification suggestion component. Based on user input, this component may suggest genetic modifications and optimize genes for desired traits. The output may be modified genetic sequences or recombinant DNA sequences. The system may include a blockchain integration component. This component may create a unique digital identity (hash) for each modified genetic sequence and record the hash on a blockchain. This may provide immutability and transparency.

In one or more embodiments, the system may include an access and distribution component. This component may allow authorized users to access the modified genetic material and facilitate collaboration and research. The output may be shared and accessible genetic information that provides proof of creation and ownership, as well as the opportunity to monetize it. The system may include additional revenue stream components. These may include an ATM machine that prints physical copies of tokens or exchanges cryptocurrency from a physical wallet to any currency. The system may also include merchandise such as physical cryptocurrency wallets with logos or unique designer pieces.

In one or more embodiments, the system may operate within a larger framework. This framework may include deep learning and manufacturing components, leading to product creation. The products may interface with software, which may connect to various elements such as DNA synthesizers and software licensing. The system may incorporate blockchain protection. It may interface with laboratories and information storage components, each potentially leading to product creation and interfacing with software. In one or more embodiments, the system may operate within a framework of policies, preservation, and regulations covering biosafety, biodiversity, cybersecurity, and intellectual property.

In one or more embodiments, the system may address several challenges in the field of synthetic biology. Conventional approaches to managing synthetic genetic codes and biological systems may lack integration between artificial intelligence, blockchain technology, and laboratory processes. This may result in inefficiencies, security vulnerabilities, and difficulties in establishing ownership and intellectual property rights for newly created genetic sequences. Traditional methods may rely on manual analysis and storage of genetic data, which may be time-consuming and prone to errors. Additionally, the lack of a secure, immutable record of genetic creations may hinder collaboration and commercialization efforts in the field of synthetic biology.

In one or more embodiments, the present disclosure provides an integrated system that combines advanced AI techniques for genetic analysis and prediction, secure blockchain technology for recording ownership and transactions, and efficient data storage solutions for managing large volumes of genetic information. The system streamlines the process of creating, analyzing, and commercializing synthetic genetic codes and biological systems while ensuring security, transparency, and proper attribution of intellectual property rights. In one or more embodiments, the system employs a novel predictive modeling platform that utilizes proprietarily trained AI combined with deep learning structures. These structures may include a Graph convolutional network, a convolutional neural network in 2D and 3D, a Recurrent neural network, and a 1D convolutional network. These networks may utilize sequential, spatial, and image data synergistically.

In one or more embodiments, the system may use neural networks, which may be a biologically-inspired programming paradigm that enables a computer to learn from observational data. The system may also use deep learning, which may be a set of techniques for learning in neural networks involving perceptrons. A perceptron may be an artificial neuron that becomes a method for weighing evidence to make decisions. In one or more embodiments, the system may include a proprietary algorithm for how blockchain works and stores information. It may have several controls and layers, with the innermost layer of genetic codes being accessible through a token purchase. In one or more embodiments, the system may utilize a digital security token backed by a real-world asset, such as newly synthesized genetic codes, sequences and biological systems. These assets may become the users'proprietary intellectual property via blockchain technology. The system may generate utility tokens at an initial coin offering (ICO) on its native blockchain network. These utility tokens may allow for services to the user. Each token generated and issued into circulation may be backed by one singular and definitive biological piece of synthesized genetic material which may have no prior existence in nature.

In one or more embodiments, the system may store models of DNA, which may save storage space. The AI software may act as a liaison between individuals/laboratories and the blockchain. The system may include built-in controls, including AI on AI and human controls, bias elimination systems, and quantum-proof algorithms. The system may be trained using various sources and robust predictive mechanisms to look for pre-selected patterns. The system may employ methods for managing synthetic genetic codes and biological systems. These methods may include analyzing genetic data using an AI component, recording transactions using a blockchain component, and storing DNA models using a data storage component.

In one or more embodiments, the method may include predicting potential applications of synthesized genetic codes. This prediction may be performed by the AI component using deep learning algorithms. The method may include validating synthetic genetic constructs. This validation may be performed by comparing the synthetic constructs against existing genetic databases and predictive models. The method may include transforming genetic sequences into digital assets. This transformation may involve creating a unique digital identifier for each genetic sequence and recording it on the blockchain. The method may include generating utility tokens on a blockchain network. These tokens may represent ownership or access rights to specific genetic information or services. The method may include eliminating biases in AI algorithms. This may be achieved through techniques such as adversarial training and regular auditing of the AI models. In one or more embodiments, the method may employ logistic regression for classification of synthetic systems. This classification may categorize synthetic systems based on various attributes such as complexity, function, or origin. The method may include cross-checking outcomes using multiple machine learning models. This cross-checking may involve comparing results from different models to increase accuracy and reliability. The method may include initiating human control for uncertain outcomes. When the AI system cannot make a definitive decision, it may flag the case for human review.

In one or more embodiments, the method may include storing DNA sequences as digital models to save storage space. This may involve compressing genetic information into more efficient digital formats. The method may include collecting genetic data from various sources. These sources may include organisms, samples, and databases. The method may include using machine learning and AI algorithms to model genetic behavior. This modeling may analyze patterns, mutations, and interactions in genetic data. The method may include suggesting genetic modifications based on user input. These suggestions may be generated by the AI component to optimize genes for desired traits. The method may include creating a unique digital identity for each modified genetic sequence. This identity may be in the form of a cryptographic hash recorded on the blockchain.

In one or more embodiments, the method may include validating modifications against original genetic material. This validation may ensure the accuracy and safety of genetic modifications. The method may include allowing authorized users to access modified genetic material. This access may be controlled through blockchain-based permissions and smart contracts. The method may include transforming DNA sequences into models for storage. This transformation may reduce storage requirements while preserving essential genetic information. The method may include creating new DNA codes or products. These creations may be generated by the AI component based on specific objectives or parameters. The method may include proofreading newly created DNA codes. This proofreading may involve checking for potential errors or unintended consequences in the genetic sequence.

In one or more embodiments, the method may include assessing the usefulness, viability, and non-harmfulness of new genetic creations. This assessment may involve predictive modeling and comparison against known genetic databases.

Although modules are dis closed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specifications. Each stage disclosed for one module may be mixed with the operational stages of another module. Each stage can be claimed on its own and/or interchangeably with other stages of other modules. The following claims will detail the operation of each module, and inter-operation between modules.

Various hardware components may be used at the various stages of operations follow the method and computer-readable medium claims. For example, although the methods have been described to be performed by a computing device, it should be understood that, in some embodiments, different operations may be performed by different networked elements in operative communication with the computing device. For example, server 110 and/or computing device 600 may be employed in the performance of some or all of the stages disclosed with regard to the methods claimed below. Similarly, apparatus 600 may be employed in the performance of some or all of the stages of the methods. As such, apparatus 105 may comprise at least those architectural components as found in computing device 600.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

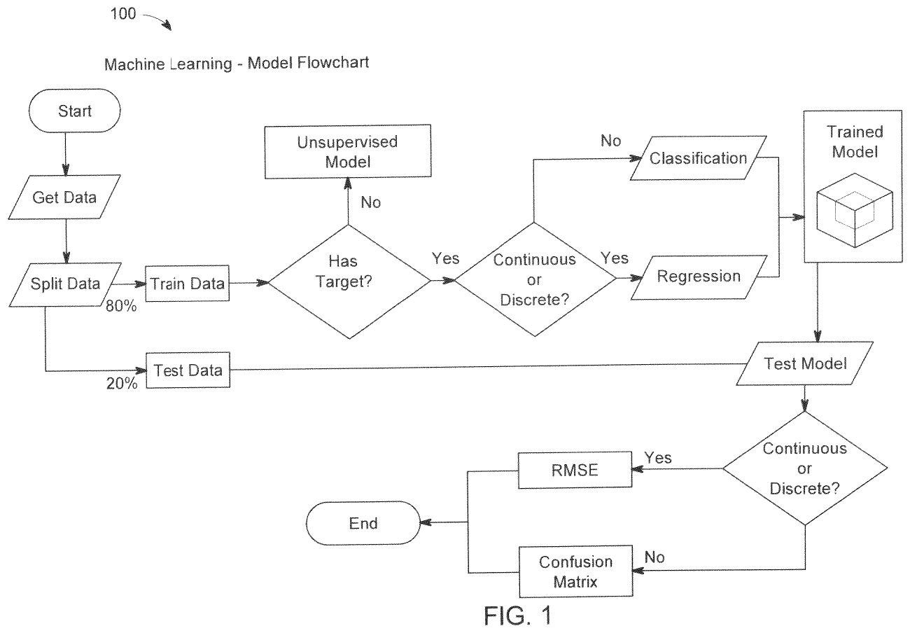

With reference now to the drawings, and in particular, FIGS. 1-6, illustrate example methods and processes to implement a system configured to

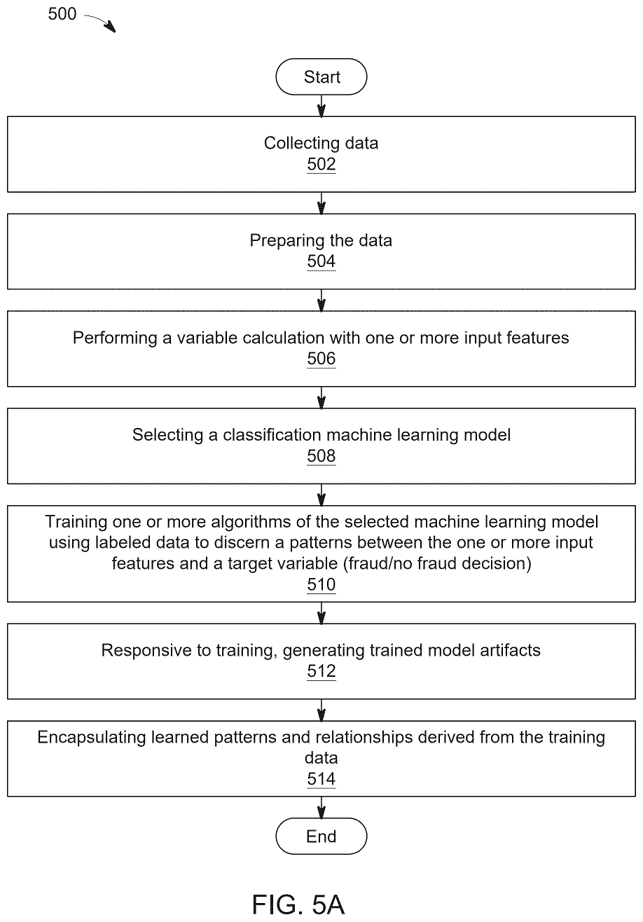

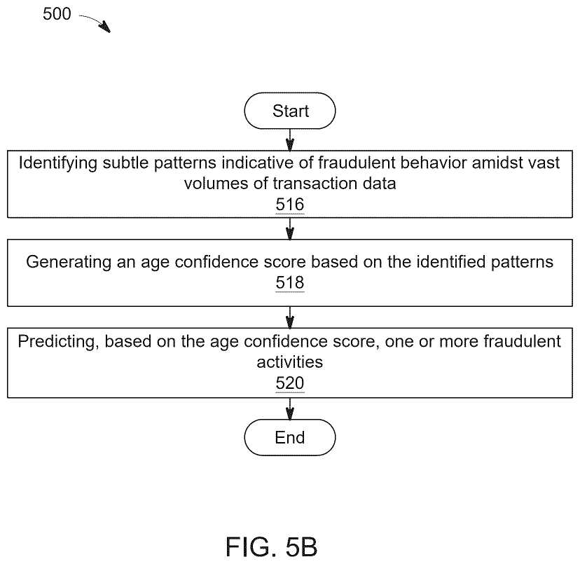

FIGS. 5A AND 5B illustrates one possible operating environment through which a platform consistent with embodiments of the present disclosure may be provided. By way of non-limiting example, a platform 150 may be hosted on a centralized server 110, such as, for example, a cloud computing service. A user may access platform 150 through a software application. The software application may be embodied as, for example, but not be limited to, a website, a web application, a desktop application, and a mobile application compatible with a computing device 600.

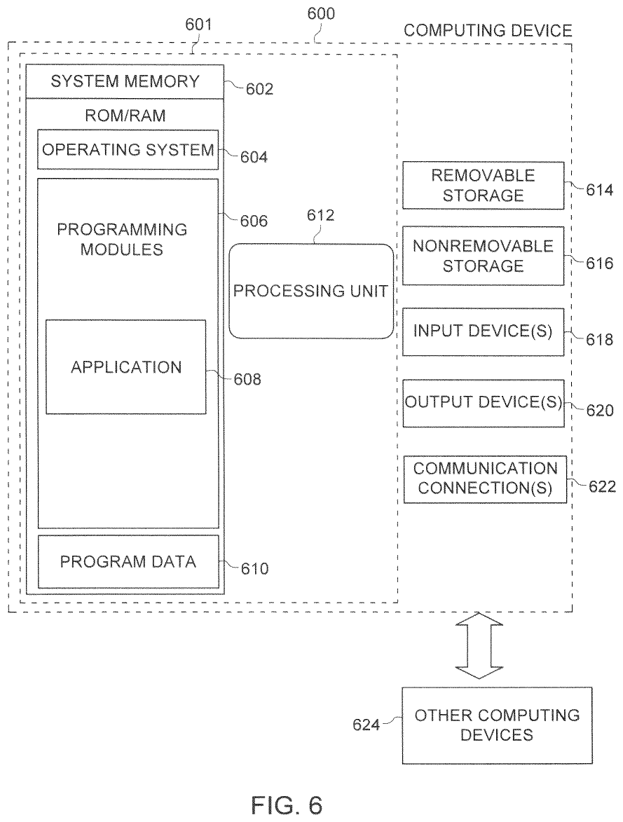

As will be detailed with reference to FIG. 6 below, the computing device through which the platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

FIG. 1 is a flow chart setting forth the general stages involved in a metho of a machine learning model 100 consistent with an embodiment of the disclosure for providing platform 150. Method 100 may be implemented using a computing device 600 as described in more detail below with respect to FIG. 6.

Although method 100 has been described to be performed by computing device 600, it should be understood that, in some embodiments, different operations may be performed by different networked elements in operative communication with computing device 600. For example, server 110 and/or computing device 600 may be employed in the performance of some or all of the stages in method 100. Moreover, server 110 may be configured much like computing device 600 and, in some instances, be one and the same embodiment. Similarly, apparatus may be employed in the performance of some or all of the stages in method 100.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 100 will be described in greater detail below.

Method 100 may begin at starting block 165 and proceed to stage 170 where computing device 600 may get data. From stage 170, method 100 may advance to stage 175 to where computing device 600 may split data. Method 100 may continue to sending a first portion of data to 180 for training and a second portion of data to 185 for testing at 220. Method 100 may proceed to a target determining stage 195 where computing device 600 may send the data to an unsupervised model 190 if it is determined the data has no target or send to 200 if it is determined to have the target. Method 100 may proceed to 205 for classification if it is determined that the data is not continuous or discrete in 200 or proceed to 210 for regression if it is determined that the data is continuous or discrete. The method 100 may proceed from 205 or 210 to trained model 215. Method 100 may proceed to test model 220. Method 100 may proceed to 230 for RMSE if it is determined that the data is continuous or discrete at 225 or proceed to 235 for a confusion matrix if it is determined that the data is not continuous or discrete. Method 100 may then end at stage 240.

FIGS. 5A AND 5B illustrates non-limiting examples of operating environments for the aforementioned modules. Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specifications. Each stage disclosed for one module may be mixed with the operational stages of another module. Each stage can be claimed on its own and/or interchangeably with other stages of other modules.

Regarding FIGS. 5A AND 5B, the platform 150 may be embodied as, for example, but not be limited to, a website, a web application, a desktop application, and a mobile application compatible with a computing device. The computing device may comprise, but not be limited to, a desktop computer, laptop, a tablet, or mobile telecommunications device. Moreover, the platform 150 may be hosted on a centralized server, such as, for example, a cloud computing service. Although method 100 has been described to be performed by a computing device 600, it should be understood that, in some embodiments, different operations may be performed by different networked elements in operative communication with computing device 600.

Embodiments of the present disclosure may comprise a system having a memory storage and a processing unit. The processing unit coupled to the memory storage, wherein the processing unit is configured to perform the stages of method 100.

FIG. 6 is a block diagram of a system including computing device 600. Consistent with an embodiment of the disclosure, the aforementioned memory storage and processing unit may be implemented in a computing device, such as computing device 600 of FIG. 6. Any suitable combination of hardware, software, or firmware may be used to implement the memory storage and processing unit. For example, the memory storage and processing unit may be implemented with computing device 600 or any of other computing devices 618, in combination with computing device 600. The aforementioned system, device, and processors are examples and other systems, devices, and processors may comprise the aforementioned memory storage and processing unit, consistent with embodiments of the disclosure.

With reference to FIG. 6, a system consistent with an embodiment of the disclosure may include a computing device, such as computing device 600. In a basic configuration, computing device 600 may include at least one processing unit 602 and a system memory 604. Depending on the configuration and type of computing device, system memory 604 may comprise, but is not limited to, volatile (e.g. random access memory (RAM)), non-volatile (e.g. read-only memory (ROM)), flash memory, or any combination. System memory 604 may include operating system 605, one or more programming modules 606, and may include a program data 607. Operating system 605, for example, may be suitable for controlling computing device 600's operation. In one embodiment, programming modules 606 may include application 620. Furthermore, embodiments of the disclosure may be practiced in conjunction with a graphics library, other operating systems, or any other application program and is not limited to any particular application or system. This basic configuration is illustrated in FIG. 6 by those components within a dashed line 608.

Computing device 600 may have additional features or functionality. For example, computing device 600 may also include additional data storage devices (removable and/or non-removable) such as, for example, magnetic disks, optical disks, or tape. Such additional storage is illustrated in FIG. 6 by a removable storage 609 and a non-removable storage 610. Computer storage media may include volatile and nonvolatile, removable and non-removable media implemented in any method or technology for storage of information, such as computer readable instructions, data structures, program modules, or other data. System memory 604, removable storage 609, and non-removable storage 610 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 600. Any such computer storage media may be part of device 600. Computing device 600 may also have input device(s) 612 such as a keyboard, a mouse, a pen, a sound input device, a touch input device, etc. Output device(s) 614 such as a display, speakers, a printer, etc. may also be included. The aforementioned devices are examples and others may be used.

Computing device 600 may also contain a communication connection 616 that may allow device 600 to communicate with other computing devices 618, such as over a network in a distributed computing environment, for example, an intranet or the Internet. Communication connection 616 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 604, including operating system 605. While executing on processing unit 602, programming modules 606 (application 620) may perform processes including, for example, one or more of method 100's stages as described above. The aforementioned process is an example, and processing unit 602 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc. Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

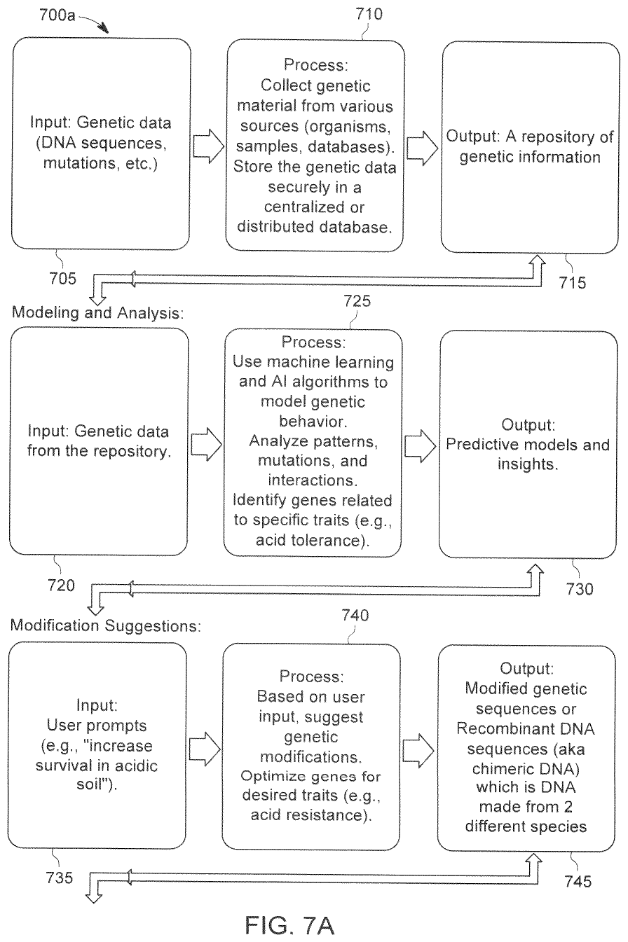

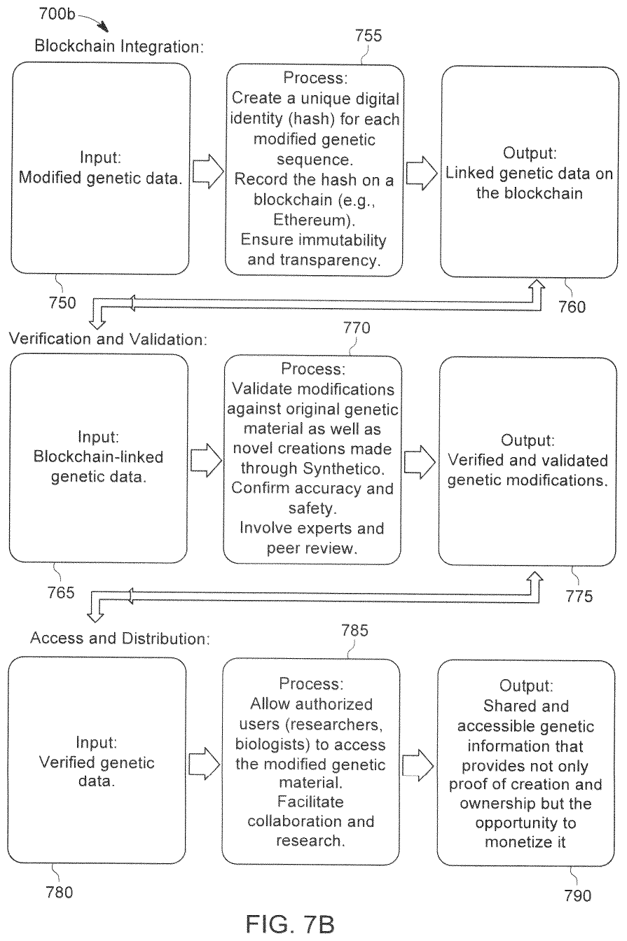

Regarding FIG. 7A, it illustrates a flowchart illustrating a process for genetic data analysis and modification. The flowchart may include three main sections: data collection and storage, modeling and analysis, and modification suggestions. The data collection and storage section may start with an input of genetic data, such as DNA sequences and mutations. The process may involve collecting genetic material from various sources and securely storing the data in a database. The output may be a repository of genetic information. The modeling and analysis section may take the genetic data from the repository as input. The process may involve using machine learning and AI algorithms to model genetic behavior, analyze patterns and interactions, and identify genes related to specific traits. The output may be predictive models and insights. The modification suggestions section may begin with user prompts as input, such as requests to increase survival in acidic soil. Based on the user input, the process may suggest genetic modifications and optimize genes for desired traits. The output may be modified genetic sequences or recombinant DNA sequences.

Regarding FIG. 7B, it illustrates a flowchart depicting three main processes: blockchain integration, verification and validation, and access and distribution. The blockchain integration process may take modified genetic data as input. The process may involve creating a unique digital identity (hash) for each modified genetic sequence and recording the hash on a blockchain. The output may be linked genetic data on the blockchain. The verification and validation process may take blockchain-linked genetic data as input. The process may involve validating modifications against original genetic material and novel creations, confirming accuracy and safety, and involving expert review. The output may be verified and validated genetic modifications. The access and distribution process may take verified genetic data as input. The process may allow authorized users to access the modified genetic material and facilitate collaboration and research. The output may be shared and accessible genetic information that provides proof of creation and ownership, as well as opportunities for monetization.

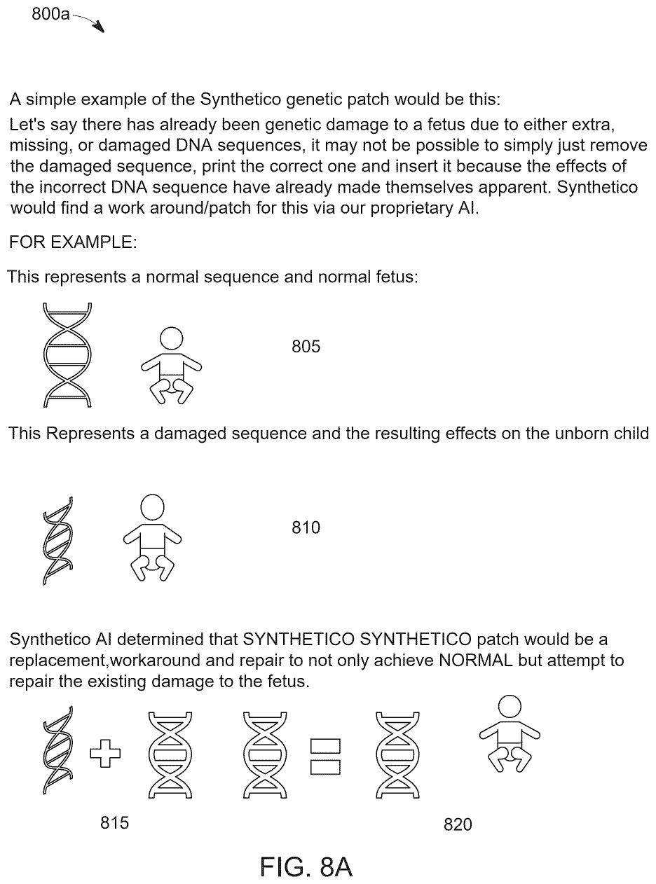

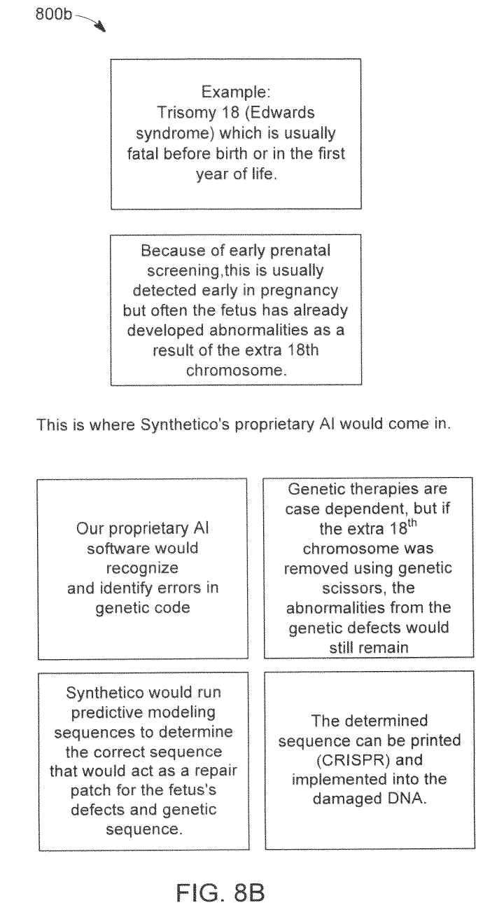

Regarding FIG. 8A, 800a it illustrates a simple example of a Synthetico genetic patch. The figure may contain three components: a representation of a normal DNA sequence and fetus 805, a representation of a damaged DNA sequence and affected fetus 810, and a representation of the Synthetico patch repairing the damaged sequence 815 and resulting in a normal fetus 820.

Regarding FIG. 8B, 800b it illustrates an example of using Synthetico's proprietary AI to address genetic disorders, specifically Trisomy 18 (Edwards syndrome). The figure may feature a series of connected text boxes and annotations showing how the AI software can recognize genetic errors, model repair sequences, and apply them through CRISPR technology.

In one or more embodiments, the system may include built-in controls to mitigate various types of biases that could affect the AI component's analysis and predictions. These controls may include AI-on-AI checks, human oversight, bias elimination systems, and quantum-proof algorithms. The system may account for and mitigate several types of biases, including but not limited to:

Implicit Bias: This refers to unconscious attitudes or stereotypes that could affect decision-making. If the training data contains implicit biases, the AI model may perpetuate those biases in its predictions. To mitigate this, the system may regularly audit and evaluate the model for bias, using techniques like debiasing algorithms and adversarial training.

Sampling Bias: This occurs when the training data is not representative of the entire population. If certain groups are underrepresented, the model may perform poorly for those groups. To mitigate this, the system may ensure diverse and balanced data collection.

Temporal Bias: This arises when the data distribution changes over time. If the model is trained on historical data, it may not generalize well to the present or future. To mitigate this, the system may regularly update the training data to reflect current trends and patterns.

Overfitting to Training Data: This occurs when the model learns noise or specific features from the training data, performing well on training data but poorly on unseen data. To mitigate this, the system may use techniques like cross-validation, regularization, and early stopping.

Edge Cases and Outliers: These are data points that deviate significantly from the norm. Ignoring or mishandling these cases can lead to biased predictions. To mitigate this, the system may explicitly consider edge cases during model development and testing.

In one or more embodiments, the AI component may employ a deep neural network topology that stacks and blends several machine learning algorithms and models. To classify various synthetic systems, the system may first employ a logistic regression algorithm to estimate binary values and solve classification problems of various synthesized categories.

The system may then employ two models that may serve as a cross-checking control mechanism: linear regression and decision trees or random forests. Linear regression, a self-learning algorithm, may establish relationships between independent and dependent variables using a y=ax+b function. This model may establish the relationship between variables and biases in the hidden layers for continuous outcomes.

Decision trees or random forests, non-parametric models, may separate information at different nodes based on different features/variables of the dataset. Outcomes from both models may be cross-checked and input into a final logistic regression control to decide on the outputs and initiate approval, disapproval, or send it to human control.

The system may implement a multi-stage classification process using a hidden layers list. This process may evaluate synthetic genetic constructs based on usefulness, viability, and potential for harm. The stages may include:
1. Sequence or complete product classification/categorization
2. Usefulness evaluation
3. Viability prediction
4. Non-harmfulness assessment

Each stage may involve multiple sub-steps and evaluations. For example, the usefulness evaluation may consider factors such as medicinal applications, research potential, and artistic value. The viability prediction may take into account environmental factors, nutritional needs, and lifecycle considerations. The non-harmfulness assessment may evaluate potential impacts on pathogens, ecosystems, and human health. The system may produce three possible outcomes: yes, no, or maybe. In cases where the AI component cannot make a definitive decision, it may flag the case for human review. This human review process may have a set timeframe, such as 7 days, with the possibility of extension. By implementing these bias mitigation strategies and multi-stage evaluation processes, the system aims to ensure accurate, fair, and safe management of synthetic genetic codes and biological systems.

Here is a detailed description of FIGS. 7A and 7B:

FIG. 7A illustrates a flowchart labeled “700A” depicting the process of genetic data management and modification. The flowchart is divided into three main horizontal sections:

### 1. Collection and Storage of Genetic Data:

705: Input-Genetic data (DNA sequences, mutations, etc.)
710: Process-Collect genetic material from various sources (organisms, samples, databases). Store the genetic data securely in a centralized or distributed database.
715: Output-A repository of genetic information

### 2. Modeling and Analysis:

720: Input-Genetic data from the repository
725: Process-Use machine learning and AI algorithms to model genetic behavior. Analyze patterns, mutations, and interactions. Identify genes related to specific traits (e.g., acid tolerance).
730: Output-Predictive models and insights

### 3. Modification Suggestions:

735: Input-User prompts (e.g., ‘increase survival in acidic soil’)
740: Process-Based on user input, suggest genetic modifications. Optimize genes for desired traits (e.g., acid resistance).
745: Output-Modified genetic sequences or Recombinant DNA sequences (aka chimeric DNA) which is DNA made from 2 different species.

FIG. 7B continues the flowchart with three additional processes:

### 4. Blockchain Integration:

Input: Modified Genetic Data
Process: Create a unique digital identity (hash) for each modified genetic sequence. Record the hash on a blockchain (e.g., Ethereum). Ensure immutability and transparency.
Output: Linked genetic data on the blockchain

### 5. Verification and Validation:

Input: Blockchain-linked genetic data
Process: Validate modifications against original genetic material as well as novel creations made through Synthetico. Confirm accuracy and safety. Involve experts and peer review.
Output: Verified and validated genetic modifications

### 6. Access and Distribution:

Input: Verified Genetic Data
Process: Allow authorized users (researchers, biologists) to access the modified genetic material. Facilitate collaboration and research.
Output: Shared and accessible genetic information that provides not only proof of creation and ownership but the opportunity to monetize it

Each process in FIG. 7B is represented by a box containing the input, process, and output stages, connected by arrows to show the flow of information and operations. This comprehensive flowchart illustrates the end-to-end process of genetic data management, from collection and analysis to modification, verification, and distribution, incorporating blockchain technology for security and traceability. Furthermore, FIG. 7B illustrates a flowchart labeled “700B” continuing the process from FIG. 7A. It is also divided into three main horizontal sections:

### 4. Blockchain Integration:

750: Input—Modified Genetic Data
755: Process—Create a unique digital identity (hash) for each modified genetic sequence. Record the hash on a blockchain (e.g., Ethereum). Ensure immutability and transparency.
760: Output—Linked genetic data on blockchain

### 5. Verification and Validation:

765: Input—Blockchain-linked genetic data
770: Process—Validate modifications against original genetic material as well as novel creations made through Synthetico. Confirm accuracy and safety. Involve experts and peer review.
775: Output—Verified and validated genetic modifications

### 6. Access and Distribution:

780: Input—Verified genetic data
785: Process—Allow authorized users (researchers, biologists) to access the modified genetic material. Facilitate collaboration and research.
790: Output—Shared and accessible genetic information. Proof of creation and ownership. Opportunity to monetize.

The blockchain integration process may begin with the modified genetic data output from the previous stage. A unique digital identity, or hash, may be created for each modified genetic sequence. This hash may then be recorded on a blockchain, such as Ethereum, to ensure immutability and transparency of the genetic modifications. The output of this stage may be genetic data that is securely linked to the blockchain.

In the verification and validation stage, the blockchain-linked genetic data may undergo a rigorous process to validate the modifications. This may involve comparing the modified sequences against the original genetic material, as well as assessing any novel creations made through the Synthetico system. The process may aim to confirm the accuracy and safety of the genetic modifications. Experts in the field and peer reviewers may be involved in this stage to ensure a thorough and unbiased assessment. The output of this stage may be a set of verified and validated genetic modifications.

The final stage in this process may focus on access and distribution of the verified genetic data. Authorized users, such as researchers and biologists, may be granted access to the modified genetic material. This stage may facilitate collaboration and research by providing a secure and transparent platform for sharing genetic information. The output of this stage may be shared and accessible genetic information that not only provides proof of creation and ownership but also offers opportunities for monetization.

Throughout these stages, the system may employ various security measures and access controls to ensure the integrity and confidentiality of the genetic data. The use of blockchain technology may provide an immutable record of all transactions and modifications, enhancing transparency and traceability in genetic research and development.

FIG. 8A, 800a depicts a simplified representation of how Synthetico's genetic patch technology may work:
1. The first image shows a purple DNA helix labeled “NORMAL” next to a simple line drawing of a baby, representing a normal genetic sequence and fetus development 805.
2. The second image shows a yellow DNA helix labeled “DAMAGED” next to a line drawing of a baby with visible differences, representing a damaged genetic sequence and resulting abnormal fetal development 810.
3. The third image illustrates the Synthetico patch process 815:
   A purple DNA helix (normal)
   Plus (+) a red DNA helix (Synthetico patch)
   Equals (=) a corrected purple DNA helix
   Followed by a line drawing of a baby with normal appearance, suggesting the patch has corrected the genetic damage and restored normal development 820.

FIG. 8B, 800b provides a more detailed flowchart of how Synthetico's proprietary AI system may address a specific genetic disorder (Trisomy 18):
1. The process starts with “Early Prenatal Screening” which detects Trisomy 18 (Edwards syndrome).
2. An arrow points to “Fetus with Extra 18th Chromosome”, noting this condition is “usually fatal before birth or in first year of life”.
3. The next step shows “Synthetico's Proprietary AI Software” which:
   Recognizes and identifies errors in genetic code
   Runs predictive modeling sequences
4 . This leads to “Determine Correct Sequence” which acts as a repair patch for:
   Fetus's genetic defects
   Genetic sequence abnormalities
5. The final step is “Print and Implement”:
   The determined sequence is printed using CRISPR technology
   The printed sequence is implemented into the damaged DNA

This flowchart illustrates how Synthetico's AI may go beyond simply removing the extra chromosome, by predicting and generating a repair sequence to address the developmental abnormalities already present in the fetus. The process aims to not only correct the chromosomal abnormality but also potentially reverse its effects on fetal development.

In one or more embodiments, the synthetic genetic code management system is provided, focusing on alternative uses and embodiments based on FIGS. 8A and 8B. In one or more alternative embodiments, the system may be applied to address specific genetic disorders detected during prenatal screening. For example, as illustrated in FIGS. 8A and 8B, the system may be utilized to address trisomy 18, also known as Edwards syndrome, which is typically fatal before birth or in the first year of life. The process may begin with early prenatal screening, which often detects trisomy 18 in the early stages of pregnancy. However, by the time of detection, the fetus may have already developed abnormalities as a result of the extra 18th chromosome. This is where the proprietary AI component of the system may provide significant value.

As shown in FIG. 8A, 800a the system may first analyze a normal DNA sequence (represented by the purple helix) and compare it to the damaged sequence found in a fetus with trisomy 18 (represented by the yellow helix). The AI component may then determine a suitable genetic patch or repair sequence. Referring to FIG. 8B, the proprietary AI software may recognize and identify errors in the genetic code. While conventional genetic therapies might simply remove the extra 18th chromosome using genetic scissors, this approach alone would not address the abnormalities that have already developed in the fetus.

To address this limitation, the system may employ its predictive modeling capabilities. As illustrated in FIG. 8B, 800b Synthetico's AI may run predictive modeling sequences to determine the correct sequence that would act as a repair patch for both the fetus's genetic sequence and the resulting physical defects. Once the appropriate repair sequence is determined, it may be synthesized using DNA printing technologies such as CRISPR. The system may then provide guidance on implementing this synthesized sequence into the damaged DNA of the fetus. In this embodiment, the system goes beyond simply removing the extra chromosome. Instead, it aims to predict and generate a repair sequence that may address the developmental abnormalities already present in the fetus. This process seeks to not only correct the chromosomal abnormality but also potentially reverse its effects on fetal development. It should be understood that while this example focuses on trisomy 18, the system may be adaptable to address a wide range of genetic disorders detected during prenatal screening. The AI component may be trained on various genetic conditions, allowing it to recognize different types of genetic errors and model appropriate repair sequences.

Furthermore, the blockchain component of the system may be utilized in this context to securely record the original genetic data, the AI-generated repair sequence, and any subsequent modifications. This may provide a transparent and immutable record of the genetic therapy process, which may be valuable for medical records, research purposes, and potential regulatory compliance. The data storage component may play a crucial role in maintaining a database of known genetic disorders, their associated sequences, and successful repair strategies. This database may continuously grow and improve as more cases are processed, potentially enhancing the system's predictive capabilities over time. In additional embodiments, the system may be extended to address genetic predispositions to certain conditions like heart disease or type 1 diabetes. The AI component may analyze genetic data to identify markers associated with these conditions and suggest potential genetic modifications to mitigate risk factors.

Moreover, the system's capabilities may be applied in the pharmaceutical industry to address medication side effects. The proprietary AI algorithms may analyze the molecular structure of drugs, determine which parts of the drug molecule are causing undesired side effects, and suggest modifications that could be integrated into the drug molecule to maintain efficacy while reducing or eliminating side effects.

Another potential application, as briefly mentioned earlier, may involve using the system to maintain biodiversity. The AI component may analyze and catalog the genetic makeup of various plants, fruits, vegetables, and animals. This information could be used to 3D print seeds or genetic material, helping to preserve the original genetic diversity that might otherwise be lost due to the increasing prevalence of genetically modified organisms (GMOs).

The system's reverse engineering capabilities may also prove valuable in various contexts. The proprietary AI algorithms may analyze biological systems and determine whether an organism or material is the result of natural mutation or artificial design. In the case of artificial design, the system may be able to reverse engineer and identify which components and underlying biological systems were used to create the de novo organism. These alternative uses and embodiments illustrate the versatility and potential impact of the synthetic genetic code management system across various fields, from prenatal care and genetic therapy to drug development, biodiversity preservation, and forensic biology. As the system continues to evolve and improve, it may open up new possibilities for addressing complex genetic and biological challenges in innovative ways.

With respect to the above description, it is to be realized that the optimum dimensional relationship for the various components of the invention described above and in the illustrations include variations in size, materials, shape, form, function, and manner of operation, assembly and use, are deemed readily apparent and obvious to one skilled in the art, and all equivalent relationships to those illustrates in the drawings and described in the specification are intended to be encompassed by the invention.

In some embodiments, certain aspects of the techniques described above may be implemented by one or more processors of a processing system executing software. The software comprises one or more sets of executable instructions stored or otherwise tangibly embodied on a non-transitory computer readable storage medium. The software can include the instructions and certain data that, when executed by the one or more processors, manipulate the one or more processors to perform one or more aspects of the techniques described above. The non-transitory computer readable storage medium can include, for example, a magnetic or optical disk storage device, solid state storage devices such as Flash memory, a cache, random access memory (RAM) or other non-volatile memory device or devices, and the like. The executable instructions stored on the non-transitory computer readable storage medium may be in source code, assembly language code, object code, or other instruction format that is interpreted or otherwise executable by one or more processors.

A computer readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computer processors, not only residing within a single machine, but deployed across a number of machines. While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

1. A system for managing synthetic genetic codes, comprising:
an artificial intelligence (AI) component;
a blockchain component; and
a data storage component.

2. The system of claim 1, wherein the AI component analyzes genetic data using deep learning algorithms.

3. The system of claim 1, wherein the blockchain component records ownership and transactions related to synthetic genetic constructs.

4. The system of claim 1, wherein the data storage component stores digital models of DNA sequences.

5. The system of claim 1, wherein the AI component employs Graph Convolutional Networks.

6. The system of claim 1, wherein the AI component employs Convolutional Neural Networks.

7. The system of claim 1, wherein the AI component employs Recurrent Neural Networks.

8. The system of claim 1, wherein the blockchain component includes a plugin for laboratory equipment wherein the plugin comprises at least one of: development and productivity plugins, web and UI design plugins, AI plugins, blockchain plugins, data analysis and visualization plugins, DevOps and automation plugins, content and media plugins, e-commerce and marketing plugins, and security and monitoring plugins.

9. The system of claim 1, wherein the blockchain component enables real-time recording of intellectual property rights.

10. The system of claim 1, wherein the blockchain component generates digital tokens representing ownership rights of synthesized genetic constructs, enabling management and monetization of genetic intellectual property.

11. A method for managing synthetic genetic codes, comprising:
analyzing genetic data using an AI component;
recording transactions using a blockchain component; and
storing DNA models using a data storage component.

12. The method of claim 11, further comprising predicting potential applications of synthesized genetic codes.

13. The method of claim 11, further comprising validating synthetic genetic constructs.

14. The method of claim 11, further comprising transforming genetic sequences into digital assets.

15. The method of claim 11, further comprising generating utility tokens on a blockchain network.

16. The method of claim 11, further comprising eliminating biases in AI algorithms.

17. The method of claim 11, further comprising employing logistic regression for classification of synthetic systems.

18. The method of claim 11, further comprising:
cross-checking outcomes using multiple machine learning models; and
initiating human control for uncertain outcomes.

19. (canceled)

20. (canceled)

21. A non-transitory computer-readable medium storing instructions that, when executed by one or more processors, cause the one or more processors to perform operations comprising:
receiving genetic data;
analyzing the genetic data using an artificial intelligence (AI) component;
recording transactions related to the genetic data using a blockchain component;
storing digital models of DNA sequences derived from the genetic data using a data storage component; and
wherein the operations further comprise at least one of the following:
predicting potential applications of synthesized genetic codes based on the analyzed genetic data;
validating synthetic genetic constructs derived from the genetic data;
transforming genetic sequences into digital assets recorded on the blockchain component;
generating utility tokens on a blockchain network associated with the blockchain component;
implementing bias elimination algorithms in the AI component to reduce biases in genetic data analysis;
employing logistic regression for classification of synthetic systems derived from the genetic data;
cross-checking outcomes using multiple machine learning models within the AI component;
initiating human control for uncertain outcomes in the genetic data analysis;
storing DNA sequences as digital models to optimize storage space in the data storage component.

22-40. (canceled)

41. The non-transitory computer-readable medium of claim 21 further comprising:
wherein the AI component is configured to perform one or more of:
identify errors in genetic code sequences;
generate repair patch sequences to correct identified genetic errors;
predict effects of applying the repair patch sequences;
analyze genetic data to detect genetic predispositions to medical conditions;
generate genetic therapy recommendations to mitigate detected predispositions;
analyze molecular structures of medications;
identify molecular components causing undesired side effects;
generate modified molecular structures to reduce identified side effects while maintaining therapeutic efficacy;
analyze biological systems to determine if an organism resulted from natural mutation or artificial design;
identify component biological systems used in the design for artificially designed organisms;
wherein the AI component employs at least one of the following:
predictive modeling to determine repair patch sequences for correcting genetic abnormalities;
machine learning models trained on genetic data to classify synthetic biological systems based on viability, usefulness, and safety;
implementing multiple layers of control, including AI-on-AI verification and human expert review, to validate classifications of synthetic biological systems:
wherein the data storage component maintains a library of original genetic sequences of plants and animals to preserve genetic biodiversity;
further comprising a synthetic biology component configured to:
receive AI-generated genetic sequences;
synthesize the genetic sequences; and
implement the synthesized sequences in target organisms.
